(19) United States
(12) Patent Application Publication
(10) Pub. No.: US 2021/0161993 A1
(43) Pub. Date: Jun. 3, 2021

(54) Method for accelerated healing of burn wounds

(71) Applicant: PVP LABS PTE. LTD., Singapore (SG)

(72) Inventors: Evgeny Alexandrovich Chertorizhsky, Belkino Village (RU); Mikhail Vladimirovich Ovchinnikov, Moscow (RU); Aleksey Viktorovich Kleimenov, Zhukov (RU)

(73) Assignee: PVP LABS PTE. LTD., Singapore (SG)

(21) Appl. No.: 17/172,533

(22) Filed: Feb. 10, 2021

Related U.S. Application Data
(63) Continuation-in-part of application No. 16/560,275, filed on Sep. 4, 2019.

(30) Foreign Application Priority Data
Sep. 12, 2018 (RU) 2018132558

(51) Int. Cl. A61K 38/08; A61K 9/00; A61K 9/08; A61K 47/38; A61K 47/22; A61P 17/02

(52) U.S. Cl. CPC A61K 38/08; A61K 9/0014; A61P 17/02; A61K 47/38; A61K 47/22; A61K 9/08

(57) ABSTRACT

There is proposed a method for accelerated healing of a burn wound by reducing thermally-induced oedema and hyperemia, normalizing a neutrophil to lymphocyte ratio, stimulating formation of fibrous components in wound-induced scar tissue. The method includes aseptic preparation of a formulation consisting of an active ingredient being hexapeptide tyrosyl-D-alanyl-glycyl-phenylalanyl-leucyl-arginine (0.02-0.5 weight %), and at least one pharmaceutically acceptable excipient making up the rest, sterile fill-finishing the formulation, quality control thereof, and noninvasive local application of the formulation after thermal injury for accelerated healing of the burn wound, as a first-aid treatment and in subsequent therapy thereof. In a preferred embodiment, the formulation is provided in the form of solution, and the pharmaceutically acceptable excipient is purified water. In another preferred embodiment, the formulation is provided in the form of gel and the at least one excipient consists of hydroxyethyl cellulose 2.5-3%, potassium sorbate 2-3% and purified water up to 100%.

### CROSS-REFERENCES TO RELATED APPLICATIONS

This U.S. patent application is a continuation-in-part application of a U.S. patent non-provisional application Ser. No. 16/560,275, published as US20200078438A1, filed on 4 Sep. 2019, entitled “Agent for the Treatment of Skin Wounds or Burns”, claiming priority under 35 U.S.C. 119 (a) through (d), under the Paris Convention, from a Russian Federation patent application RU2018132558 filed 12 Sep. 2018, now Patent of Russian Federation RU2687485, hereby entirely incorporated by reference.

### FIELD OF THE INVENTION

The invention pertains to medicine and concerns a method for accelerated healing of burn wound through the reduction of thermally-induced oedema and hyperemia, normalization of the neutrophil to lymphocyte ratio, stimulation of the formation of fibrous components in wound-induced scar tissue, based on application of hexapeptide tyrosyl-D-alanyl-glycyl-phenylalanyl-leucyl-arginine.

### BACKGROUND OF THE INVENTION

According to World Health Organization (WHO), burns are a global public health problem, as an estimated 180 000 deaths annually are caused by burns (see Reference [1] at the end of present description). It is also known that non-fatal burns are a major cause of morbidity, long-term hospitalization and persistent disability, and they are often accompanied by social stigma and significantly degrade the quality of life (Reference [1]). The principle of unity of topical and systemic therapy that underpins the treatment of burn patients places special emphasis on topical treatment, which is aimed at thorough healing of burn wounds and early restoration of the skin (Reference [2]). The development of new effective topical burn treatments therefore remains an important objective for public health.

Thermal injury is associated with an entire complex of pathophysiological mechanisms, such as hypoxia and lipid peroxidation (LPO), reduction of tissue perfusion in the zone of stasis, neutrophil aggregation and hyperactivation, a cascade of inflammatory reactions and severe pain. New products must obviously rely on a multifaceted mechanism of action, which is aligned with the pathogenetic aspects of injury. From this perspective, clinical development of a new method for treatment of burns on the basis of a synthetic regulatory peptide with a wide spectrum of biological activity represents considerable interest.

As a component of the stress-limiting system, hexapeptide tyrosyl-D-alanyl-glycyl-phenylalanyl-leucyl-arginine (INN: tyrosyl-D-alanyl-glycyl-phenylalanyl-leucyl-arginine, also known as pharmaceutical drug Dalargin) exhibits antioxidant properties (Reference [3]), inhibits LPO (Reference [4]) and improves peripheral microcirculation (Reference [5]).

Despite being a mediator of the opioid antinociceptive system, it is almost completely incapable of crossing the blood-brain barrier. It predominantly exhibits its analgesic effect at the peripheral/tissue level, which makes it possible to consider this product as having some properties of a topical anaesthetic (Reference [6]).

Additionally, this hexapeptide also showed serious promise with respect to tissue regeneration. Specifically, several researchers demonstrated that it stimulated mitotic activity in cells by interacting with their opioid receptors and activated wound repair mechanisms (References [7, 8, 9]).

This hexapeptide in the form of medicinal product Dalargin was authorized for human use in Russia for the treatment of peptic ulcer disease and pancreatitis as a product reducing the acidity of gastric juice and exocrine activity of the pancreas (Reference [10]). However, the efficacy of this drug in therapy of peptic ulcer disease is also largely due to its reparative effect on gastric and duodenal mucosa (References [11, 12, 13]). The efficacy of Dalargin as part of combination therapy of Crohn's disease (IM 1 mg twice a day) (patent RU2363455 filed 2007 Nov. 19) is also suggestive of a systemic reparative effect. The work of E. V. Maksakova suggested that the wound healing potential of Dalargin should be used in the treatment of corneal injuries (Reference [14]).

The “decision-making process” happening at the cellular level (apoptosis or DNA synthesis and mitosis) largely depends on oxidative status or redox status (Reference [15]), which is especially important for reducing the zone of stasis of burn wounds. Antioxidant properties, antiradical and nitrergic activity (Reference [16]) obviously give Dalargin additional capabilities related to regeneration in burn wounds.

An invention in the field of cosmetology was published, in which Dalargin was used as a component of an anti-inflammatory cream for alleviating the symptoms of acne (patent RU 2045949, 1995). Another patent RU2221547 claimed immunomodulating activity of Dalargin as a component of a cosmetic product.

Data of an experiment are available, which demonstrated in vitro that Dalargin, when added to peripheral blood samples of burn patients (n=15), normalized elevated functional and metabolic activity of neutrophils, reduced elevated activity of NADPH oxidase, alkaline phosphatase (ALP), myeloperoxidase and nuclear chromatin and normalized elevated levels of cationic proteins in neutrophils and the total number of activated neutrophils. Importantly, in the blood of healthy humans (n=27), Dalargin had an opposite effect of increasing the total number of activated neutrophils and NADPHase activity. These effects were mediated by opioid receptors and were dose-dependent, with maximum effect being achieved at a molar concentration of 5-10-s M (Reference [17]). However, this effect was not studied in burn models in vivo.

In general, any tissue injury results in neutrophil activation, which leads to increased production of active oxygen radicals, LPO and triggers a whole cascade of reactions that damage tissues spared by the pathological process (Reference [18]). Therefore, the ability of Dalargin to normalize neutrophil hyperactivation is a pathogenetically important link in its burn healing effect.

Results of experiments investigating the efficacy of parenteral administration of Dalargin for burn treatment have been published, but burn healing activity of the drug when used as a topical application has not been addressed in any of the studies. One work compared Dalargin with delta sleep-inducing peptide (DSIP) in burn treatment in rats; both products were administered to test animals intraperitoneally (patent RU2070054C1, publication date 1996 Dec. 10). The efficacy of Dalargin on wound healing process is not however disclosed in RU2070054C1. The authors reported an increased mortality rate (75%) in the group of rats with intraperitoneal administration of Dalargin in dose of 0.5 mg/kg body weight during 14 days after burn injury, compared to 60% in control group (rats with no treatment after burn injury) and 20% in group with DSIP administration after burn injury. In another study, Dalargin (0.3 mg subcutaneously (SC)) was associated with significant acceleration of skin repair and reduced intensity of productive inflammation in a 3rd degree burn model in rats (Reference [9]). Patent RU2196603C2 discloses the use of Dalargin (1-2 mg in 1-2 mL by IV infusion) for the treatment of burns as part of infusion therapy.

Unlike the works cited, the present invention discloses a method for accelerated healing of the burn wound after local noninvasive administration of Dalargin through the reduction of thermally-induced oedema and hyperemia, normalization of the neutrophil to lymphocyte ratio, stimulation of the formation of fibrous components in wound-induced scar tissue.

Additionally, there are known data concerning the use of Dalargin in a number of other diseases. Patents UA 6829U, UA 6823U and UA 6826U disclose the use of Dalargin for the treatment of acute experimental pancreatitis. The use of Dalargin as an anti-stress agent was covered in patents UA 67632, UA 67630 and UA 67629 in experimental models of chronic pancreatitis, acute adnexitis and peritonitis respectively. The anti-stress activity of Dalargin was demonstrated in patent UA 67626 in an experimental chronic stress model. Patent RU 2180598 discloses the use of Dalargin for the treatment of toxic hepatitis in patients with chronic drug addiction. The efficacy of Dalargin in the treatment of viral diseases was demonstrated in patents RU 2261722 (treatment of a latent form of genital herpes in a woman with the foetal loss syndrome) and RU 2167671 (treatment of tick-borne encephalitis). Publication MD1413F discloses the use of Dalargin for the treatment of the oral mucosa and lichen planus, and publication MD1296F presents data on the therapy of lichen planus with Dalargin. Patent RU 2008131509 presents a pharmaceutical composition for the treatment of demyelinating diseases, which includes Dalargin. Patent RU 2218896 discloses the use of Dalargin for the treatment of bullous keratopathy. Publications MD1963F and MD1610F cover the use of Dalargin in cases of chronic recurrent oral ulceration, and patent RU 2230549 covers its use for the treatment of allergic dermatosis. A publication by A. V. Dontsov (Reference [19]) demonstrated the efficacy of Dalargin for cytokine profile correction in patients with ischaemic heart disease and metabolic syndrome. At the same time, the present invention, while claiming the efficacy of Dalargin-based method for burn therapy, is fundamentally different from the above cited works, which pertain to the treatment of other diseases.

To summarize the above, it can be concluded that Dalargin shows great promise from the standpoint of clinical development, and its multifaceted activity, which eliminates pathophysiological processes (oxidative stress, LPO, hyperinflammatory and nociceptive response) and stimulates reparative processes, and is responsible for the undeniable advantages of the proposed method for accelerated healing of the wound bed through the reduction of thermally-induced oedema and hyperemia, normalization of the neutrophil to lymphocyte ratio, stimulation of the formation of fibrous components in wound-induced scar tissue.

The closest analogue of the claimed invention is a method of administration of D-Panthenol, a topical medication with wound- and burn-healing properties that contains per 1 g: as the active ingredient: dexpanthenol: 50 mg; excipients: phenonip: 4 mg; lanolin: 160 mg; white beeswax: 50 mg; soft white paraffin: 210.8 mg; dimethicone: 5 mg; Lanette SX emulsifier: 20 mg; propylene glycol: 20 mg; butylhydroxyanisole: 0.1 mg; butylhydroxytoluene: 0.1 mg; decamethylcyclopentasiloxane: 20 mg; magnesium sulfate heptahydrate: 5 mg; protegin W: 295 mg; purified water: 160 mg (described in RLS, entry for D-Panthenol. Last updated by the manufacturer on 2020 Nov. 16, <www.rlsnet.ru/tn_index_id_12869.htm>).

The active ingredient of this product is dexpanthenol, which works by stimulating skin regeneration, normalizing cell metabolism and increasing the strength of collagen fibers. An obvious disadvantage of this medicinal product lies therefore in the fact that it does not involve other biological mechanisms that are activated in cases of skin injury and participate in wound healing.

### SUMMARY OF THE INVENTION

In view of the above, the goal of the invention is to develop a method for accelerated healing of the burn wound through the reduction of thermally-induced oedema and hyperemia, normalization of the neutrophil to lymphocyte ratio, stimulation of the formation of fibrous components in wound-induced scar tissue. Essential features of the claimed invention at least are:
production of a formulation for noninvasive local administration of tyrosyl-D-alanyl-glycyl-phenylalanyl-leucyl-arginine for stimulating tissue regeneration processes in the wound bed, reducing the severity of productive inflammation after thermal injury and reducing oedema and hyperaemia;
a pronounced local wound and burn accelerated healing effect, which results from Dalargin's multifaceted activity involving elimination of pathophysiological processes (oxidative stress, LPO, hyperinflammatory and nociceptive response) and enhancement of reparative processes, ensuring undeniable advantages in the local therapy of skin wounds and burns.

Being charged, large and hydrophilic, proteins are poor permeators (of a poor bioavailability) (as described in Reference [21]). Therefore, in formulating dosage forms of peptides drugs, an intelligent selection of additives which enhance their absorption across membranes and their stability is very significant (Reference [21]). In preferred embodiments, the formulation for the hexapeptide noninvasive delivery for treatment of skin wounds or burns consists of an active ingredient in the form of hexapeptide tyrosyl-D-alanyl-glycyl-phenylalanyl-leucyl-arginine at 0.02-0.5 weight % of the formulation, and pharmaceutically acceptable excipients making up the rest of the formulation.

### IN SPECIFIC EMBODIMENTS

the inventive formulation is a solution, wherein the pharmaceutically acceptable excipient is purified water; and
the inventive formulation is a gel, wherein the pharmaceutically acceptable excipients are: hydroxyethyl cellulose 2.5-3%, potassium sorbate 2-3% and purified water up to 100%, in weight percentage of the formulation.

For achieving this goal, there is proposed a method for accelerated healing of the burn wound, based on preparation and noninvasive local application of a dosage form containing, as the active ingredient, hexapeptide tyrosyl-D-alanyl-glycyl-phenylalanyl-leucyl-arginine at 0.02-0.5 weight %, with pharmaceutically acceptable excipients (purified water, hydroxyethyl cellulose, potassium sorbate) making up the rest.

In a particular embodiment of the invention, the method for accelerated healing of the burn wound is based on the preparation and noninvasive local application of a solution containing tyrosyl-D-alanyl-glycyl-phenylalanyl-leucyl-arginine and a pharmaceutically acceptable excipient at the following ratio of the ingredients in weight %: hexapeptide tyrosyl-D-alanyl-glycyl-phenylalanyl-leucyl-arginine: 0.02-0.5 weight %, purified water up to 100%.

In a particular embodiment of the invention, the method for accelerated healing of the burn wound is based on the preparation and local application of a gel containing tyrosyl-D-alanyl-glycyl-phenylalanyl-leucyl-arginine and pharmaceutically acceptable excipients at the following ratio of the ingredients in weight %: hexapeptide tyrosyl-D-alanyl-glycyl-phenylalanyl-leucyl-arginine: 0.02-0.5%, hydroxyethyl cellulose 2.5-3%, potassium sorbate 2-3%, purified water up to 100%.

### BRIEF DESCRIPTION OF THE DRAWINGS

FIG. 1 illustrates an average healing rate of burn wounds (cm²/day), where I=control, II=Dexpanthenol, III=hexapeptide tyrosyl-D-alanyl-glycyl-phenylalanyl-leucyl-arginine (0.02-0.5% solution).

FIG. 2 illustrates lymphocyte-neutrophil ratio vs. time after thermal injury, where the vertical axis represents the ratio, and the horizontal axis represents follow-up days; Group I=control, Group II=Dexpanthenol, Group III=hexapeptide tyrosyl-D-alanyl-glycyl-phenylalanyl-leucyl-arginine (0.02-0.5% solution).

FIG. 3 illustrates effect on skin wound healing, where the horizontal axis represents follow-up days and the vertical axis represents the mean linear dimension of the wound; Group I=control, Group II=Dexpanthenol, Group III=hexapeptide tyrosyl-D-alanyl-glycyl-phenylalanyl-leucyl-arginine (0.02-0.5% solution).

FIG. 4 illustrates an average healing rate of burn wounds (cm²/day), where Group I=control, Group II=Bepanthen, Group III=hexapeptide tyrosyl-D-alanyl-glycyl-phenylalanyl-leucyl-arginine (0.02% gel), Group IV=hexapeptide tyrosyl-D-alanyl-glycyl-phenylalanyl-leucyl-arginine (0.5% gel).

FIG. 5 illustrates the following tables:

Table 1. Burn wound oedema score vs. time as a measure of response to thermal injury, where * marks statistically significant differences relative to the control group, & marks statistically significant differences relative to the dexpanthenol group.

Table 2. Burn wound hyperaemia score vs. time, where * marks statistically significant differences relative to the control group, ^{&} marks statistically significant differences relative to the dexpanthenol group.

FIG. 6 illustrates the following tables:

Table 3. Planimetric evaluation results (hexapeptide tyrosyl-D-alanyl-glycyl-phenylalanyl-leucyl-arginine in the form of a solution): burn area (in cm²), where * marks statistically significant differences relative to the control group, ^{&} marks statistically significant differences relative to the dexpanthenol group.

Table 4. Planimetric evaluation results (hexapeptide tyrosyl-D-alanyl-glycyl-phenylalanyl-leucyl-arginine in the form of a gel): burn area (in cm²), where * marks statistically significant differences relative to the control group, ^{&} marks statistically significant differences relative to the Bepanthen group.

### PREFERRED EMBODIMENTS OF THE INVENTION

### Example 1

### Preparation of Formulation for Hexapeptide Noninvasive Application in the Form of a Topical Solution for Accelerated Healing of Wounds

(a) For the preparation of a topical solution, a predetermined quantity of tyrosyl-D-alanyl-glycyl-phenylalanyl-leucyl-arginine diacetate (0.04-1.0 g) is added to 200 mL of purified water under slow mixing at 22° C.;
(b) the solution obtained in step (b) is mixed at 22-25° C. until completely dissolved, avoiding overheating;
(c) the resulting solution obtained in step (c) is passed through a sterile filter (pore size 0.22 μm) to produce a sterile tyrosyl-D-alanyl-glycyl-phenylalanyl-leucyl-arginine diacetate solution for accelerated healing of wounds; the product is filled and sealed in sterile containers; and
(d) the sterile tyrosyl-D-alanyl-glycyl-phenylalanyl-leucyl-arginine diacetate solution must be stored in a dark cold place (at +2° C. to +10° C.), avoiding freezing.

Content of the Solution for Topical Use:

| Name | Content |
|---|---|
| Hexapeptide tyrosyl-D-alanyl-glycyl- | 0.02-0.5 g |
| phenylalanyl-leucyl-arginine diacetate | |
| Purified water | up to 100 mL |

### Example 2

### Preparation of Formulation for Tyrosyl-D-Alanyl-Glycyl-Phenylalanyl-Leucyl-Arginine Noninvasive Application in the Form of Gel for Accelerated Healing of Wounds

For the preparation of 150 mL of gel for healing of burn injuries, aseptic manufacturing and sterile fill-finish is used as follows:

(a) a reactor is charged with 135-139 mL of purified water, the mixing is turned on and powdered components are added: 0.04-0.75 g of hexapeptide tyrosyl-D-alanyl-glycyl-phenylalanyl-leucyl-arginine, 4-4.5 g of hydroxyethyl cellulose, and 3-4 g of potassium sorbate;
(b) all the components are mixed at 25° C. for 30 minutes until a homogeneous tyrosyl-D-alanyl-glycyl-phenylalanyl-leucyl-arginine diacetate-containing emulsion is obtained;
(c) the emulsion obtained in step (b) is sterilized by passage through a sterile 0.45 μm membrane filter;
(d) the emulsion gel obtained in step (c) is filled and sealed under aseptic conditions in containers;
(e) the sterile tyrosyl-D-alanyl-glycyl-phenylalanyl-leucyl-arginine diacetate gel must be stored in a dark cold place (at +2° C. to +10° C.), avoiding freezing.

Content of Gel for Accelerated Healing of Burn Wounds:

| Name | Content, weight % |
|---|---|
| Hexapeptide tyrosyl-D-alanyl-glycyl- | 0.02-0.5 |
| phenylalanyl-leucyl-arginine | |
| Hydroxyethyl starch | 2.5-3 |
| Potassium sorbate | 2-3 |
| Purified water | up to 100 |

### Example 3

### Results of Experiment to Evaluate the Efficacy of Hexapeptide Tyrosyl-D-Alanyl-Glycyl-Phenylalanyl-Leucyl-Arginine in Burn Model in Rats

The efficacy of topical administration of tyrosyl-D-alanyl-glycyl-phenylalanyl-leucyl-arginine in the therapy of thermal burns was evaluated on models of II-IIIA degree burns in 30 outbred male rats. The animals were kept in a vivarium (ambient temperature 19-23.degree. C.) according to Good Laboratory Practices of the Eurasian Economic Union and the applicable guidelines (Reference [20]).

Flat-bottom glass containers of an appropriate diameter filled with liquid paraffin to ⅔ of their volume were used as the thermal agent. The containers were heated on a water bath before application to the skin of the rats, which were randomized to three groups (n=10 in each group): the control group and two experimental groups. Another tissue repair stimulator, dexpanthenol (5% topical ointment), was used as the comparator. All the rats received burns with a standardized area of ≈40 cm². Within 20 min. after inflicting the thermal injury, control group animals were treated by applying an aseptic dressing followed by therapy with Iodopiron and Levomecol (group I); animals in the experimental groups additionally received dexpanthenol (group II) or 0.02-0.5% solution of hexapeptide tyrosyl-D-alanyl-glycyl-phenylalanyl-leucyl-arginine (group III consisting of 5 animals that received 0.02% solution and 5 animals that received 0.5% solution).

The experiment involved evaluating the general condition of the animals and performing gross examination to evaluate the severity of tissue oedema in the burn region, hyperaemia, infiltration, appearance of wound discharge and wound epithelialization. These signs were scored as follows: maximum severity of a sign corresponded to a score of 3 points, and absence of a sign to a score of 0. Objective assessment of the healing process was based on planimetric measurements. All the observations were performed on days 1, 3, 5, 7 and 10 of the experiment.

The healing rate was determined using the formula V=(S1−S2)/t, where S1=wound area during the previous measurement, S2=wound area during the next measurement, t=number of days between the previous and the next measurement.

The experiment also included an evaluation of the white blood cell population as a ratio of the total number of lymphocytes to segmented cells in venous blood.

Results. On day 1 of the experiment, the general condition of all the test animals was evaluated as moderate, which was due to the extent of the inflicted thermal injury. The animals in the cages exhibited little movement, slept most of the time and weakly responded to external stimuli.

By day 3 their activity and mobility increased, the animals exhibited a defensive response when taken out of the cages, developed appetite, etc. No intergroup differences in the general condition of the animals were observed. Table 1 lists oedema scores of the burn wound bed and adjacent tissues. Analysis of the results indicates that the initial oedema as a response to thermal injury and the time of its disappearance were significantly less in the group receiving hexapeptide tyrosyl-D-alanyl-glycyl-phenylalanyl-leucyl-arginine both relative to the control group and to the Dexpanthenol group. For example (Table 1), for day 1: Group I (control)/Group II (Dexpanthenol)/Group III (hexapeptide tyrosyl-D-alanyl-glycyl-phenylalanyl-leucyl-arginine) show the following numbers: 3.0±0.3/2.0±0.5/1.0±0.4. For day 5: Group I/Group II/Group III show the following numbers: 0.9±0.3/0.7±0.5/0.3±0.2. For day 7: Group I/Group II/Group III show the following numbers: 0.3±0.3/0.3±0.3/0.0±0.0. Thus, Group III heals much faster than Groups I and II.

Similar dynamics were also observed in the case of hyperaemia (Table 2). The treatments reduced skin erythema already on day 1. Meanwhile, the application of hexapeptide tyrosyl-D-alanyl-glycyl-phenylalanyl-leucyl-arginine was statistically significantly more effective to the one of Dexpanthenol in reducing hyperaemia. Moreover, burn wound hyperaemia score reached 0 already on day 5 after thermal injury in tyrosyl-D-alanyl-glycyl-phenylalanyl-leucyl-arginine group, compared to day 7 for Dexpanthenol group.

Planimetry demonstrated pronounced statistically significant positive dynamics of burn wound healing in Group III animals (who received tyrosyl-D-alanyl-glycyl-phenylalanyl-leucyl-arginine). It was characterized by significant reduction of burn area already on day 3 of the experiment, reduction of burn depth and, consequently, more favorable course of the wound healing process. Results obtained in Groups II and III also demonstrated the superiority of applying hexapeptide tyrosyl-D-alanyl-glycyl-phenylalanyl-leucyl-arginine (Group III) for reducing the area of burn injury (Table 3). Thus, Group III heals much faster than Groups I and II.

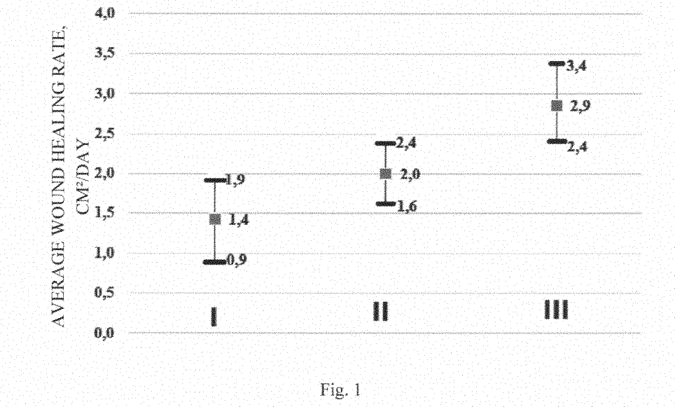

The average healing rate is shown in FIG. 1. Statistically significant differences were only detected between the control Group I and Group III receiving hexapeptide tyrosyl-D-alanyl-glycyl-phenylalanyl-leucyl-arginine.

Change of the lymphocyte-neutrophil ratio over time was analyzed. Considering that one distinguishing trait of rats as a biological model is the predominance of monocytes in the white blood cell population of the peripheral blood (with lymphocytes normally making up 65-77% of all white blood cells in healthy animals), the change in white blood cell profiles in the study groups was investigated by evaluating the lymphocyte-neutrophil ratio (the ratio of lymphocytes to segmented cells). Neutrophil-lymphocyte ratio in patients with burn injury is used as a prognostic value for the diagnosis of sepsis and bacteraemia (Reference [22]).

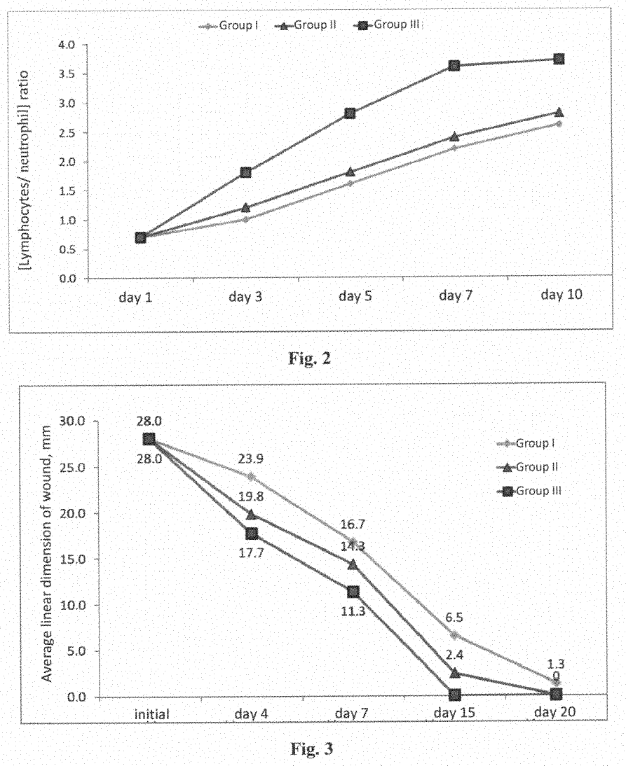

Analysis demonstrated that on day 1 the lymphocyte-neutrophil ratio was substantially reduced, indicating the expected response to thermal injury. Changes of the lymphocyte-neutrophil ratio over time, which are shown in FIG. 2, exhibit statistically significant differences between Group I (control) and Group III (hexapeptide tyrosyl-D-alanyl-glycyl-phenylalanyl-leucyl-arginine). In Group III this parameter returned to normal already on day 7, while in the other groups it continued to exhibit an upward trend.

To summarize, topical administration of tyrosyl-D-alanyl-glycyl-phenylalanyl-leucyl-arginine immediately after thermal injury allows to reduce oedema and hyperaemia. It also demonstrates pronounced efficacy in terms of wound surface reduction, average wound healing rate and dynamics of the lymphocyte-neutrophil ratio. In other words, this means that Group III is healed much faster than Groups I and II.

The results obtained in the preclinical experiment provide evidence of the high efficacy of the use of hexapeptide tyrosyl-D-alanyl-glycyl-phenylalanyl-leucyl-arginine both as a first-aid treatment and in the subsequent therapy of moderate surface burns. Additionally, the results justify the statement that hexapeptide tyrosyl-D-alanyl-glycyl-phenylalanyl-leucyl-arginine can also be used for wound bed preparation in cases of deep burns for subsequent skin transplantation and in the treatment of trophic ulcers in weakened patients with a low regenerative potential.

### Example 4

### Results of Experiment to Evaluate the Efficacy of Hexapeptide Tyrosyl-D-Alanyl-Glycyl-Phenylalanyl-Leucyl-Arginine in Planar Wound Model in Rats

The study was performed in white male Wistar rats. All the animals were randomized to three groups of 13 rats each: (I) control Group (untreated), (II) comparator Group (Bepanthen topical cream with 5% dexpanthenol) and (III) experimental Group (hexapeptide tyrosyl-D-alanyl-glycyl-phenylalanyl-leucyl-arginine). The Group III consisted of 5 animals that received 0.02% solution and 5 animals that received 0.5% solution.

Linear wounds were modeled by an approximately 4-cm-long longitudinal incision in the skin and subcutaneous fat along the midline of the back. After making the incision, the edges of the wound were brought together, and three sutures were applied at an equal distance. The animals received the study drugs starting on the day of the operation; the control Group received a placebo. In each group, 3 rats were intended for a tensometric study, which required euthanasia on day 7 of the experiment. Samples for a histological evaluation were collected immediately after complete healing (on days 25-26).

The study drugs were applied once daily, every day, for 20 days. The wound healing effect was evaluated based on the clinical course (presence of purulent discharge, dynamics of wound edge attachment) on days 4, 7, 15 and 20 of the experiment.

FIG. 3 presents results of visual inspection that clearly demonstrate the more rapid healing of the linear wound in the Group III (hexapeptide tyrosyl-D-alanyl-glycyl-phenylalanyl-leucyl-arginine) relative to the control Group I and active comparator Group II. Thus, Group III is healed much faster than Groups I and II in this example as well.

Healing days 15-20, which correspond to the third phase of healing, are known to be marked by epithelialization and scar formation. However, healing in the control Group was not complete even on day 20 of observation, the scab remained, and complete epithelialization in this group occurred only on days 25-26.

Based on microscopic examination results, collagen fibers in the intact zone in the control Groups were loosely arranged and thick, whereas in the wound healing region collagen fibers were much thinner, and a large number of capillaries remained, i.e. the scar exhibited multiple signs of immaturity. Additionally, epithelial regeneration in the wound edge consolidation zone was not complete. Focal and diffuse inflammatory infiltrates were observed in the scar structure. Granulation tissue extended deep in to the dermis, almost to the boundary of subcutaneous fat.

In Group III receiving hexapeptide tyrosyl-D-alanyl-glycyl-phenylalanyl-leucyl-arginine, the scar ended at the depth of hair follicles; emptying of the blood vessels was observed, and fibrous components were dominant in scar tissue relative to cellular components, which indicates that the scar was at the concluding phase of development.

Intermediate results of histological evaluation (between the control Group I and Group III receiving hexapeptide tyrosyl-D-alanyl-glycyl-phenylalanyl-leucyl-arginine) were obtained in Group II receiving therapy with Bepanthen cream.

Results of tensiometric evaluation demonstrated substantial (over than twofold) superiority of Group III relative to the control Group I.

### Example 5

### Results of Experiment to Evaluate the Efficacy of Hexapeptide Tyrosyl-D-Alanyl-Glycyl-Phenylalanyl-Leucyl-Arginine in the Form of 0.02% and 0.5% Gel in Burn Model in Rats

The efficacy of topical administration of hexapeptide tyrosyl-D-alanyl-glycyl-phenylalanyl-leucyl-arginine in the therapy of thermal burns was evaluated on models of II-IIIA degree burns in 40 outbred male rats. The animals were kept in a vivarium according to Good Laboratory Practices of the Eurasian Economic Union and the applicable guidelines (Reference [20]). Flat-bottom glass containers of an appropriate diameter filled with liquid paraffin to ⅔ of their volume were used as a thermal agent. The animals were randomized to four groups (n=10 in each group): Group I was the control group. A popular tissue repair product, Bepanthen (topical cream with 5% dexpanthenol), was used as the comparator. Burn area was standardized at Æ40 cm².

An aseptic dressing applied within the first 20 minutes after the burn, followed by therapy with Iodopiron and Levomecol, was used for control Group I. Group II was treated with Bepanthen. Group III received hexapeptide tyrosyl-D-alanyl-glycyl-phenylalanyl-leucyl-arginine as 0.02% topical gel, while Group IV received hexapeptide tyrosyl-D-alanyl-glycyl-phenylalanyl-leucyl-arginine as 0.5% topical gel.

In addition to monitoring the general condition of the animals on days 1, 3, 5, 7 and 10 of the experiment, the healing process was evaluated planimetrically. The healing rate was determined using the formula V=(S1-S2)/t, where S1=wound area during the previous measurement, S2=wound area during the next measurement, t=number of days between the previous and the next measurement.

Results. On day 1 of the experiment, the general condition of all of the test animals was moderate. Their condition improved and activity increased by day 3 of the experiment. No differences in the general condition of the animals between the study groups were observed at this stage.

Planimetric findings are presented in Table 4. There were a pronounced statistically significant positive dynamics of burn wound healing in Groups III and IV (hexapeptide tyrosyl-D-alanyl-glycyl-phenylalanyl-leucyl-arginine 0.02% and 0.5% respectively). It was characterized by significant (both relative to the control Group and the Bepanthen Group) reduction of burn area already on day 3 of the experiment, reduction of burn area and depth and, consequently, more favorable course of the wound healing process. No statistically significant differences were detected between Groups III and IV, although Group IV demonstrated numerically greater reduction of burn area relative to Group III (Table 4). Thus, Groups III and IV are healed much faster than Groups I and II in this experiment too.

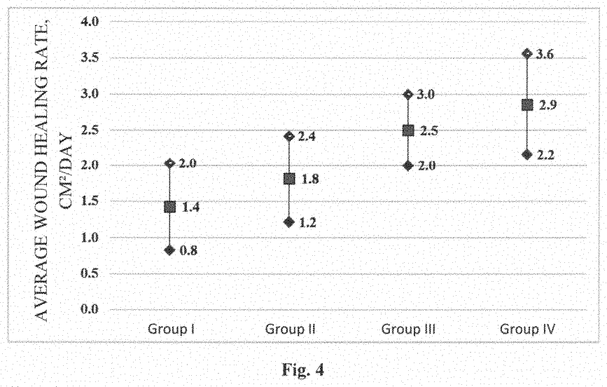

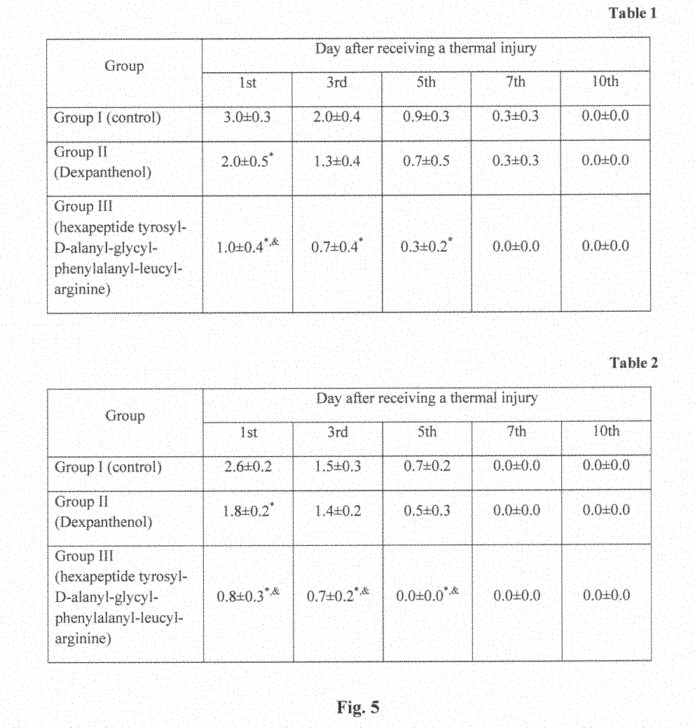

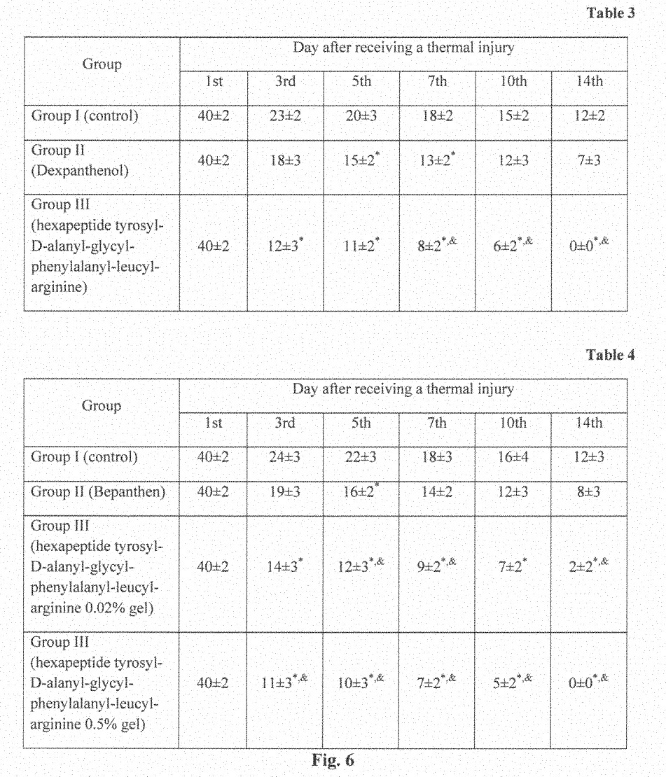

The average healing rate is shown in FIG. 4. Statistically significant differences were only detected between the control Group I and Groups III and IV receiving the hexapeptide tyrosyl-D-alanyl-glycyl-phenylalanyl-leucyl-arginine in gel form (both 0.02% and 0.5%). Despite the fact that wound healing parameters in Group IV were better, no significant differences relative to Group III were detected owing to a small sample size in this study. The results confirm pronounced efficacy of the gel containing 0.02% or 0.5% of hexapeptide tyrosyl-D-alanyl-glycyl-phenylalanyl-leucyl-arginine with respect to reducing burn surface area and improving the average healing rate of burn wounds.

The results obtained in the preclinical experiment provide evidence of the high efficacy of hexapeptide tyrosyl-D-alanyl-glycyl-phenylalanyl-leucyl-arginine both as a first-aid treatment and in the subsequent therapy of moderate surface burns. The results justify the statement that hexapeptide tyrosyl-D-alanyl-glycyl-phenylalanyl-leucyl-arginine can be used in a topical gel, or topical solution dosage forms for wound bed preparation in cases of deep burns for subsequent skin transplantation and in the treatment of trophic ulcers in patients with a low regenerative potential. The therapeutic range of concentrations for hexapeptide tyrosyl-D-alanyl-glycyl-phenylalanyl-leucyl-arginine in the topical solution or topical gel dosage forms is 0.02-0.5%.

### REFERENCES

[1] World Health Organization Fact Sheets, 6 Mar. 2018, <https://www.who.int/news-room/fact-sheets/detail/burns>.
[2] A. A. Alekseyev. Modern methods of burn and burn disease treatment (Sovremennyye metody lecheniya ozhogov i ozhogovoy bolezni)//Kombustiologiya (e-version).—1999.—1.
[3] A. N. Zokhirov et al. Effect of Dalargin, a Synthetic Analogue of Opioid Peptides, on the Anti-oxidant Status of Dogs//Vestnik Kurskoy Gosudarstvennoy Selskokhozyaystvennoy Akademii.—2016.—9.
[4] S. P. Lvova, T. F. Gorbunova, E. M. Abayeva. Effect of Hypothermia and Dalargin on Lipid Peroxidation in Rat Tissues (Vliyaniye gipotermii i dalargina na perekisnoye okisleniye lipidov v tkanyakh krys)//Voprosy Meditsinskoy Khimii.—1993.—Vol. 39.—3.—p. 21-24.
[5] K. V. Zhmerenetski. Microcirculation and the Effects on it of Pharmaceutical Drugs of Different Classes in Cardiovascular Diseases (Mikrotsirkulyuatsiya i vliyaniye na neyo lekarstvennykh preparatov raznykh klassov pri serdechnososudistykh zabolevaniyakh). Dr. Med. Sci. Dissertation.—2013.
[6] I. B. Zabolotskikh, S. V. Chuprin, A. N. Kurzanov. Dose-dependent Effects of Dalargin in Anaesthesiology and Intensive Care (Dozozavisimiye effekty dalargina v anesteziologii i intensivnoy terapii)//Vestnik Intensivnoy Terapii.—2002.—4.—p. 75-79.
[7] A. B. Shekhter et al. Effect of the Opiopeptide Dalargin on Reparation Processes during Wound Healing (Vliyaniye opiopeptida dalargina na reparativniye protsessy pri zazhivlenii ran)//Byull. eksperimentalnoy biologii i meditsiny.—1988.—10.—p. 487-490.
[8] T. D. Pankova, S. S. Timoshin. Evidence of the Stimulating Effect of Dalargin on the Cell Division Process being Realised through Opioid Receptors (Dokazatelstva realizatsii stimuliruyushchego effekta dalargina na protsess kletochnogo deleniye cherez opiatniye retseptory)//Byul. eksper. biol. i med.1990.—7.—p. 96-98.
[9] A. A. Noskov et al. Effect of Dalargin on skin regeneration after a burn in rats (Vliyaniye preparata dalargin na regeneratsiyu kozhi posle ozhoga u krys)//Aktualniye Voprosy Meditsiny v Sovremennykh Usloviyakh.—2017.—p. 59-60.
[10] Instruction for use of the medicinal product for human use Dalargin-Deko (LP-004596, web portal of the State Registry of Medicinal Products <https://grls.rosminzdrav.ru/>, accessed 31 Jan. 2018).
[11] S. S. Timoshin, S. A. Alekseyenko, A. A. Shtuka. Effect of Dalargin on the Repair Capability of the Gastroduodenal Mucosa in Duodenal Ulcer Patients (Vliyaniye dalargina na reparativnuyu sposobnost gastroduodenalnoy slizistoy obolochki u bolnykh yazvennoy boleznyu dvenadsatiperstnoy kishki)//Klinicheskaya Meditsina-1991.—Vol. 69.—3.—p. 75-77.
[12] S. A. Alekseyenko et al. Effect of Dalargin on the Repair Capability of the Gastrointestinal Mucosa in Various Gastrointestinal Diseases (Vliyaniye dalargina na reparativnuyu sposobnost slizistoy obolochki zheludochno-kishechnogo trakta pri razlichnykh gastroenterologicheskikh zabolevaniyakh)//Dalnevostochny Meditsinsky Zhurnal.—2010. 3.
[13] V. A. Vinogradov, V. M. Polonsky, V. G. Smagin. Effect of Dalargin on Repair Processes in the Mucosa of the Gastroduodenal Region (Vliyaniye dalargina na reparativniye protsessy v slizistoy obolochke gastro-duodenalnoy zony)//Byull VKNTs.—1982.—⁵.—p. 40-42.
[14] E. V. Maksakova. Dalargin in the Treatment of Corneal Trauma (Dalargin v lechenii travmaticheskikh povrezhdeniy rogovitsy)//Oftalmolog. Zhurnal-2000.—⁶.—p. 95-97.
[15] D. K. Das, N. Molik. Transformation of the Death Signal into the Survival Signal in Redox Signalling (Prevrashcheniye signala gibeli v signal vyzhivaniya pri redoks-signalizatsii)//Biokhimiya.—2004.—Vol. 69 No. 1. p. 16-24.
[16] E. Yu. Zhivotova, O. A. Lebedko, S. S. Timoshin. Effect of Structural Analogues of Leu-Enkephalin on DNA Synthesis Processes and Free Radical Oxidation in the Gastric Mucosa of White Mice (Vliyaniye strukturnykh analogov ley-enkefalina na protsessy sinteza DNK i svobodnoradikalnoye okisleniye v slizistoy obolochke zheludka belykh krys)//Dalnevostochny Meditsinsky Zhurnal.—2012.—1.
[17] B. V. Balachevsky, A. N. Kurzanov, A. A. Slavinsky. Dalargin-induced Modulation of Functional-Metabolic Activity of Neutrophilic Leukocytes//Uspekhi Sovremennogo Yestestvoznaniya.—2008.—5.
[18] V. I. Dontsov et al. Active Forms of Oxygen as a System: Significance for Physiology, Pathology and Natural Ageing (Aktivniye formy kisloroda kak sistema: znacheniye v fiziologii, patologii i estestvennom starenii)//Trudy ISA RAN.—2006.—Vol. 19.—p. 50-69.
[19] A. V. Dontsov. Efficacy of Dalargin for the Correction of Cytokine Profile in IHD and Metabolic Syndrome Patients (Effektivnost dalargina v korrektsii tsitokinovogo profilya u bolnykh IBS i metabolicheskim sindromom)//Kursky Nauchno-Praktichesky Vestnik Chelovek i Ego Zdorovye.—2013.—1.
[20] Guidelines on the Clinical Studies of Medicinal Products. Part one./ed. A. N. Mironov. Moscow. Grif i K, 2012.—944 p.
[21] Jitendra, Sharma P K, Bansal S, Banik A. Noninvasive routes of proteins and peptides drug delivery. Indian J Pharm Sci. 2011; 73(4):367-375. doi:10.4103/0250-474X.95608.
[22] Fuss J, Voloboyeva A, Poliovyj V. Prognostic value of using neutrophil-lymphocyte ratio in patients with burn injury for the diagnosis of sepsis and bacteraemia. Pol Przegl Chir. 2018 Jun. 15; 90(5):13-16. doi: 10.5604/01.3001.0012.0971. PMTD: 30426944.

We claim:

1. A method for accelerated healing of a burn wound of a subject, by reducing thermally-induced oedema and hyperemia, normalizing a neutrophil to lymphocyte ratio, stimulating formation of fibrous components in wound-induced scar tissue, wherein said method comprises the steps of:
(a) an aseptic preparation of a formulation consisting of:
an active ingredient in the form of hexapeptide tyrosyl-D-alanyl-glycyl-phenylalanyl-leucyl-arginine or its pharmaceutically acceptable salt at 0.02-0.5 weight % and
at least one pharmaceutically acceptable excipient making up the rest of the formulation;
(b) sterile fill-finishing of the formulation obtained in step (a);
(c) quality control of the formulation obtained in step (b); and
(d) noninvasive local application of the formulation obtained in step (c), after thermal injury of the subject for accelerated healing of the burn wound as a first-aid treatment and in subsequent therapy thereof.

2. The method according to claim 1, wherein said formulation is provided in the form of solution, and said at least one pharmaceutically acceptable excipient is purified water.

3. The method according to claim 1, wherein said formulation is provided in the form of gel, and said at least one pharmaceutically acceptable excipient consists of hydroxyethyl cellulose 2.5-3%, potassium sorbate 2-3% and purified water up to 100%.
